(19) United States
(12) Patent Application Publication
(10) Pub. No.: US 2019/0255040 A1
(43) Pub. Date: Aug. 22, 2019

(54) COMPOSITION AND METHOD FOR TOPICAL JS-K AS THERAPY FOR ACTINIC KERATOSIS

(71) Applicant: JSK THERAPEUTICS, INC, Sandy, UT (US)

(72) Inventor: Thomas P. Kennedy, Sandy, UT (US)

(21) Appl. No.: 16/279,713

(22) Filed: Feb. 19, 2019

Related U.S. Application Data
(60) Provisional application No. 62/631,928, filed on Feb. 18, 2018.

(51) Int. Cl. A61K 31/495; A61K 9/06; A61K 9/00; A61P 17/12; A61P 35/00

(52) U.S. Cl. CPC A61K 31/495; A61K 9/06; A61P 35/00; A61P 17/12; A61K 9/0014

(57) ABSTRACT

Provided herein is a composition and method for the treatment of a skin condition including actinic keratosis and cancer, the composition comprising O2-(2,4-dinitrophenyl)1-[(4-ethoxycarbonyl)piperazin-1-yI]diazen-1-ium-1,2-diolate, (JS-K) in various formulations.

### CROSS-REFERENCES TO RELATED APPLICATIONS

This application is a continuation-in-part of and claims priority to U.S. Provisional Patent Application No. 62/631,928 entitled “TOPICAL JS-K AS THERAPY FOR ACTINIC KERATOSIS and filed on Feb. 18, 2018 for Thomas P. Kennedy, which is incorporated herein by reference.

### BACKGROUND OF THE INVENTION

### FIELD OF THE INVENTION

This invention relates to treatment of skin cancer precursors and more particularly relates to treatment for actinic keratosis.

### DESCRIPTION OF THE RELATED ART

Actinic keratosis is one of the most common lesions with malignant potential to arise in the skin. It occurs in fair skinned persons with sun exposure. In Australia, the country with the highest skin cancer rate, the prevalence of actinic keratosis is 40-60% in adults over 40 years of age. In the US, the prevalence in whites is 11-26% of older adults. Clinically, actinic keratoses range from rough spots of skin to elevated hyper-proliferative plaques several centimeters in diameter on a red base. Over time a small percentage can progress primarily to squamous cell cancer of the skin. Treatment with 5-flourouracil (5-FU), photodynamic therapy (PDT) or the toll-like receptors 7/8 agonist imiquimod is inflammatory and painful. The nonsteroidal diclofenac is not painful but is much less effective. A more effective and less inflammatory and painful therapy is needed. Beneficially, such a therapy would promote the healing of actinic keratosis and prevent development of associated skin cancers including squamous cell cancer.

### SUMMARY OF THE INVENTION

The present invention has been developed in response to the present state of the art, and in particular, in response to the problems and needs in the art that have not yet been fully solved by currently available therapies. Accordingly, the present invention has been developed to provide a therapy that overcomes many or all of the above-discussed shortcomings in the art.

Provided herein is a composition for the treatment of a skin condition including actinic keratosis and cancer, the composition comprising O2-(2,4-dinitrophenyl)1-[(4-ethoxycarbonyl)piperazin-1-yI]diazen-1-ium-1,2-diolate, (JS-K) In some embodiments the composition further comprises a lipophilic carrier. The concentration of JS-K in the composition may be in the range from about 0.5 mg/mL to about 25 mg/mL and is sometimes 12 mg/mL.

In certain embodiments the lipophilic carrier comprises a hydrocarbon, a vegetable oil, an animal fat, a silicone, an alcohol, an acid, a combination of lipophilic excipients, a self-emulsifying formula, a polyglycolyzed glyceride, a polymeric excipient, a surfactant, a pluronic micellar formulation, a cyclodextrin, a polyoxyether, or other carriers.

The composition herein may be formulated as a lotion, a gel, a powder, a paste, an ointment, a wax, an oil, a lipid, a lipid containing vesicle, an anhydrous absorption paste, an oil-in-water or water-in-oil emulsion, a carbowax, a polyethelene glycol, a lipophilic skin emollient, a penetration enhancer, a semisolid gel or a semi-solid mixture.

The composition sometimes comprises a sustained-release formula. In certain embodiments the sustained-release formula comprises semipermeable matrices of solid hydrophobic polymers which may comprise a shaped article, a film, a microcapsule, a polyester, a hydrogel, a copolymer of L-glutamic acid and gamma ethyl-L-glutamate, a non-degradable ethylene-vinyl acetate, or a degradable lactic acid-glycolic acid copolymer.

Further provided herein is a method for the treatment of a skin condition, the method comprising applying to a patient in need of treatment a composition comprising O2-(2,4-dinitrophenyl) 1-[(4-ethoxycarbonyl)piperazin-1-yI] diazen1-ium-1,2-diolate, designated herein as JS-K. The daily dose of JS-K may be in the range of about .5 mg to about 200 mg. In some embodiments the of administration of JS-K is topical, transdermal, cutaneous, subcutaneous, mucosal, or transmucosal. The daily dose of JS-K may be administered once daily or two or more times daily. The daily dose is sometimes administered as a sustained release formula.

In some embodiments the skin condition treated is actinic keratosis or cancer. The cancer treated may be squamous cell skin cancer, basal cell carcinoma, Merkel cell carcinoma, lyphoma of the skin, and/or melanoma skin cancer.

The patient in need of treatment may be a mammal, a bird, a reptile, an amphibian, or a fish. In some embodiments the mammal is a human.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims or may be learned by the practice of the invention as set forth hereinafter.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a scatter plot illustrating the growth of human squamous skin carcinoma cells (HatCat 5K-Cells [10 μM]) over a two-day period;

FIG. 2A is a bar graph depicting a count of Sunburned cells for treated and untreated mice exposed to UV light;

FIG. 2B is a bar graph showing the percent of treated and untreated cells positive for 8-oxoguanine;

FIG. 2C is a bar graph showing comparative thickness of treated and untreated epidermal cells.

### DETAILED DESCRIPTION OF THE INVENTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. One skilled in the relevant art will recognize that the invention can be practiced without one or more of the specific details, or with other therapies, compounds, materials, and so forth. In other instances, well-known structures, materials, or operations are not shown or described in detail to avoid obscuring aspects of the invention.

Provided herein are a composition and method for treating actinic keratosis (AK) and squamous cell skin cancer (SCC) using O2 (2,4-dinitrophenyl) 1-[(4-ethoxycarbonyl)piperazin-1-yI]diazen-1-ium-1,2-diolate, (JS-K). In some embodiments the JS-K is formulated in a lipophilic topical cream.

The JS-K may be formulated in a lipophilic carrier, including any excipient, or combination of excipients, that enhances the solubility of a lipophilic drug (e.g., JS-K) above the solubility of that drug in water. The following listing of excipients and vehicles for solubilization and formulation of lipophilic (aka, hydrophobic) drug substances are examples and are not all inclusive or limiting:

Hydrocarbons including petrolatum, paraffin wax, liquid paraffin and mineral oil, microcrystalline wax, plastibase (Jelene), ceresin, white/yellow soft paraffin, carnauba wax;

Vegetable oils and animal fats including coconut oil, bees wax (white or yellow), olive oil, lanolin (anhydrous and hydrous), peanut oil, spermaceti wax, sesame oil, almond oil, castor oils, cotton seed oils, soy bean oils, corn oils, grape seed oils, and hydrogenated and/or sulfated derivatives of these oils;

Alcohols, acids, and esters including cetyl alcohol, cetostearyl alcohol, cetomacrogol 1000, stearic acid, stearyl alcohol, oleic acid, oleyl alcohol, palmitic acid, lauryl alcohol, lauric acid, myristyl alcohol, ethyl oleate, isopropyl myristate, lanolin alcohol;

Silicones including Dimethylpropylsiloxanes, methyl phenyl polysiloxanes, steryl esters of dimethyl polysiloxanes;

Other carriers including cocoyl caprylocaprate, coco-caprylate-caprate (Cetiol LC PH); Cetyl Palmitate 15 (Cutine CP PH), decyl oleate (Cetion V PH), isopropyl myristate, oleyl alcohol (HD Eutanol V PH); octyldodecanol (Eutanol G PH); triglycerides medium chain (Myritol 318 PH), sucrose acetate isobutyrate; propylene glycol; polyethylene glycols, polyoxy-35-castor oil, polyethoxylated castor oil, polyethoxylated 12-hydroxystearic acid;

Combinations of individual lipophilic excipients to form lotions and semi-solid creams and ointments; creams (oil-in-water emulsions where the lipophilc internal phase serves as the solubilizing phase for lipophilic drugs), ointments (water-in-oil emulsions where the lipophilic external phase serves as the solubilizing phase for lipophilic drugs);

Self emulsifying dosage forms (SEDDS) and self microemulsifying dosage forms (SMEDD);

Polyglycolyzed glycerides, for example Labrasol (saturated polyglycolysed (PEG-8) caprylic/capric glycerides), Labrafac CC (caprylic acid 54.4%, capric acid 44.8%), and Labrafil M 1944CS(mixture of oleic acid 62.65%, linoleic acid 26.7%, palmitic acid 4.74%, and mono- and di-fatty acid esters of PEG-6);

Polymeric excipients that self-associate to form micelles or semi-solid gel type structures with domains of relative hydrophobicity and hydrophilicity, for example, triblock and diblock polymers comprised of relatively hydrophobic blocks (e.g., poly lactide-co-glycolide) and hydrophilic blocks (e.g., polyethylene oxide and polyethylene glycol) often found as A-B-A or A-B block copolymers and polymeric surfactants such as poloxamers (Pluronics) or poloxamines (Tetronics);

In some embodiments the carrier comprises one or more surfactant. Surfactants possess both hydrophilic and hydrophobic functional groups. When combined with the excipients and dosage forms listed above the surfactants serve as either direct solubilization enhancing agents or as stabilizing agents and manufacturing aids for other self-associated phases as found in colloids, particularly in emulsions (including creams and ointments). Surfactants (ionic and non-ionic) may increase solubility of hydrophobic drugs. Examples include polysorbates (Tweens), sorbitan esters (Spans), Brij and Myrj surfactants, polymeric surfactants (ionic and non-ionic) and ionic surfactants (e.g., sodium lauryl sulfate) that are common surfactants in pharmaceutical formulations and are used to increase solubility of hydrophobic drugs.

The carrier is sometimes a timed-release formula. The patient response may be evaluated by observation or other methods. In certain embodiments the dosage and formulation is customized to the specific patient.

### Mechanism

One of the major consequences of chronic skin exposure to sunlight is overt skin cancer. Skin cancer is produced by Ultra Violet B (UVB) light in the three steps of initiation, promotion, and progression. UVB acts independently as a carcinogen. First, upon sun exposure UVB initiates mutagenic changes in DNA, resulting in permanent alteration in the keratinocyte. Second, chronic exposure to the UVB in sunlight results in epigenetic changes that promote the clonal expansion of initiated cells and over many years causes premature hyperplastic cutaneous growths called actinic keratoses. Third, in progression an important minority of these lesions undergo malignant conversion to basal or squamous cell skin cancers. Some investigators consider actinic keratosis to be a form of early squamous cell skin cancer.

Nitric oxide (NO) is a natural free radical regulatory signaling molecule.

The novel NO donor JS-K is a first-in-class cancer chemotherapeutic agent. JS-K releases NO upon interaction with the cell antioxidant glutathione (GSH) in a reaction catalyzed by the enzyme Glutathione S-Transferase (GST). This reaction exploits the presence of heightened GST activity levels in malignant cells compared to normal cells. JS-K is active in models of acute myelogenous leukemia (AML), multiple myeloma (MM), prostate cancer, lung cancer, liver cancer, brain cancer, Ewing's sarcoma, and glioblastoma, and inhibits metastasis in kidney cancer. JS-K acts to inhibit growth of cancer cells by multiple mechanisms including generation of the reactive nitrogen species NO, which can combine with H2O2 to form the highly reactive and mutagenic molecule peroxinitrite (ONOO).

### Unexpected Effect

Actinic keratosis and subsequent squamous skin cancers are produced by the continual mutagenic effects of radiation induced reactive oxygen and nitrogen species. Thus, it is unexpected that JS-K, a NO donor, is a useful treatment for actinic keratosis (AK) and squamous cell skin cancer (SCC). In certain embodiments Actinic keratoses are treated in an effort to prevent subsequent development of skin cancer.

The NO donor DETANONOate has previously been shown to radiosensitize cells but has not been previously suggested or shown to be effective by itself as a potential treatment for skin cancer or actinic keratosis. Overproduction of NO has also been suggested as pathogenic in hyper-proliferative skin diseases and skin tumor progression. Furthermore, UV light induces local skin production of NO, and sunscreens reduce UV light induced production of NO as a major mechanism by which they inhibit photocarcinogenesis. Thus, the known art teaches away from the direct use of an NO releasing compound such as JS-K to treat AK and SC.

### Formulation and Dosage

In various embodiments the NO donor O2-(2,4-dinitrophenyl)1-[(4 ethoxycarbonyl)piperazin-1-yl]diazen-1-ium-1,2-diolate, or JS-K, is used to treat actinic keratosis and squamous cell skin cancer. JS-K may be formulated in a pluronic micellar formulation using polyoxyethers such as P123. JS-K may also be directly formulated in lipophilic skin emollients and penetration enhancers. A topical lipophilic formulation of NO donor O2-(2,4-dinitrophenyl) 1-[(4 ethoxycarbonyl)piperazin-1 -yl]diazen-1-ium-1,2-diolate, or JS-K, may be used to treat skin conditions including but not limited to Basal cell carcinoma, Merkel cell carcinoma, Lyphoma of the Skin, and Melanoma Skin Cancer

The composition and method may be administered under physician prescription or over the counter. The route of administration is in accord with known methods including without limitation; transdermal, cutaneous, subcutaneous, mucosal, transmucosal, or by sustained release systems as noted below.

An effective amount of composition to be employed therapeutically will depend, for example, upon the specific composition, therapeutic objectives, the route of administration, and the stage and extent of the actinic keratosis. Accordingly, the therapist may titer the dosage as required to obtain the optimal therapeutic effect. The clinician may administer the composition until a dosage is reached that achieves the desired effect. The progress of this therapy may be monitored by conventional assays or by the assays described herein.

Briefly, dosage formulations of the compounds described herein are prepared for storage or administration by mixing the compound having the desired degree of purity with physiologically acceptable carriers, excipients, or stabilizers, for example Cyclodextrin and gamma-Cyclodextrin. Such materials are non-toxic to the recipients at the dosages and concentrations employed, and may include buffers such as TRIS HC1, phosphate, citrate, acetate and other organic acid salts, counterions such as sodium and/or nonionic surfactants such as TWEEN, PLURONICS, or polyethyleneglycol.

Suitable examples of sustained-release preparations include semipermeable matrices of solid hydrophobic polymers containing the composition provided, which matrices are in the form of shaped articles, films or microcapsules. Examples of sustained-release matrices include polyesters, hydrogels (e.g., poly(2-hydroxyethyl-methacrylate), copolymers of L-glutamic acid and gamma ethyl-L-glutamate, non-degradable ethylene-vinyl acetate, degradable lactic acid-glycolic acid copolymers such as poly-D-(-)-3-hydroxybutyric acid. While polymers such as ethylene-vinyl acetate and lactic acid-glycolic acid enable release of molecules for over 100 days, certain hydrogels release proteins for shorter time periods.

The dosage of the composition herein for a given patient will be determined by the therapist or physician taking into consideration the natural molecule comprising the composition and various factors known to modify the action of drugs including severity and type of disease, route of administration, age, weight, health, and other factors of the patient, other medications and other relevant clinical factors. Therapeutically effective dosages may be determined by either in vitro or in vivo methods.

Dosage may range from less than .5 mg to more than 200 mg of JS-K. In some embodiments the dosage is from 1 to 5 mg, from 5 to 10 mg, from 10 to 20 mg, from 20 to 30 mg, from 30 to 40 mg, from 40 to 50 mg, from 50 to 60 mg, from 60 to 70 mg, from 70 to 80 mg, from 80m to 90 mg, from 90 to 100 mg, from 100 to 125 mg, from 125 to 150 mg, from 150 to 1785 mg, or from 175 to 200 mg of JS-K. The JS-K may be administered as a topical gel or by other avenues described herein or known in the art. In certain embodiments the dose may be administered once daily or more frequently. In some embodiments the dose is administered twice daily, once in the AM and once in the PM. The clinician may administer the therapeutic composition as provided herein until a dosage is reached that achieves the desired effect. The progress of this therapy may be monitored by observation, conventional assays or specialized assays.

It will be appreciated that administration of therapeutic entities in accordance with the compositions and methods herein may be administered with suitable carriers, excipients, and other agents that are incorporated into formulations to provide improved transfer, delivery, tolerance, and the like. These formulations include, for example, powders, pastes, ointments, jellies, waxes, oils, lipids, lipid (cationic or anionic) containing vesicles (such as LIPOFECTIN®), anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present composition, provided that the active ingredient in the formulation is not inactivated by the formulation and the formulation is physiologically compatible and tolerable with the route of administration and as known in the art.

The embodiments may be practiced in other specific forms. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

### EXAMPLES

### Example 1: Suppression of the Growth of Human Squamous Skin Carcinoma Cells

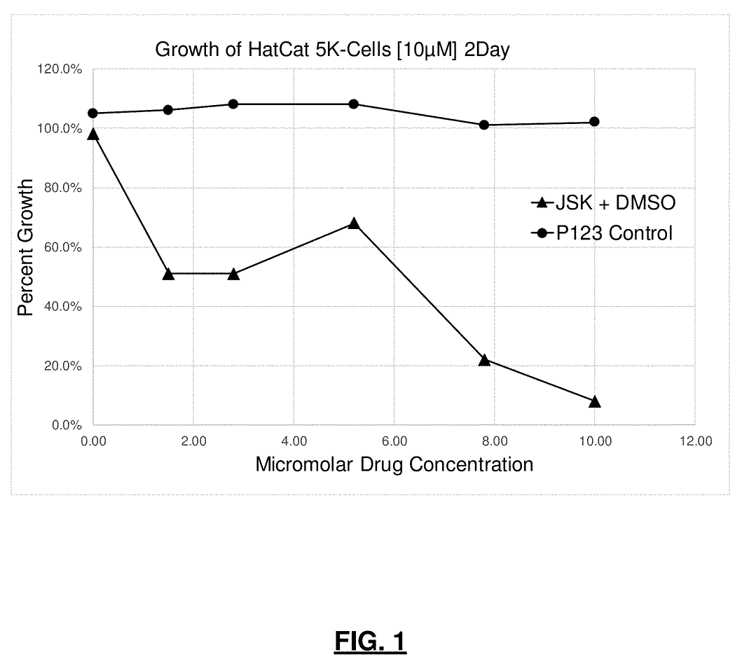

Referring to FIG. 1, JS-K at 12 mg/mL in 200 mL of acetone was active in suppressing the growth in culture of human squamous skin carcinoma Cells (HatCat cells) with IC50 concentrations <1000 nM, compared to no effect from dimethylsulfoxide (DMSO) or P1223 pluronic micellesAfd.

### Example 2: Animal Efficacy for UV Damage

Eight week-old female SKH1 mice were used. There were 5 mice in the control (no treatment, no UV) group and 10 mice in each treatment group. Treated mice all received one exposure of 600 Joules/m2 of UVB.

Immediately after UVB treatment, mice in each group were treated as follows: 200 uL of acetone only; JS-K at a concentration of 12 mg/mL in 200 uL of acetone; JS-K at a concentration of 6 mg/mL in 200 uL of acetone. Treatments were started immediately after UVB exposure and given twice (8 hours apart) on Day 1 and Day 2. On Day 3, mice were sacrificed and skin collected for histologic analysis.

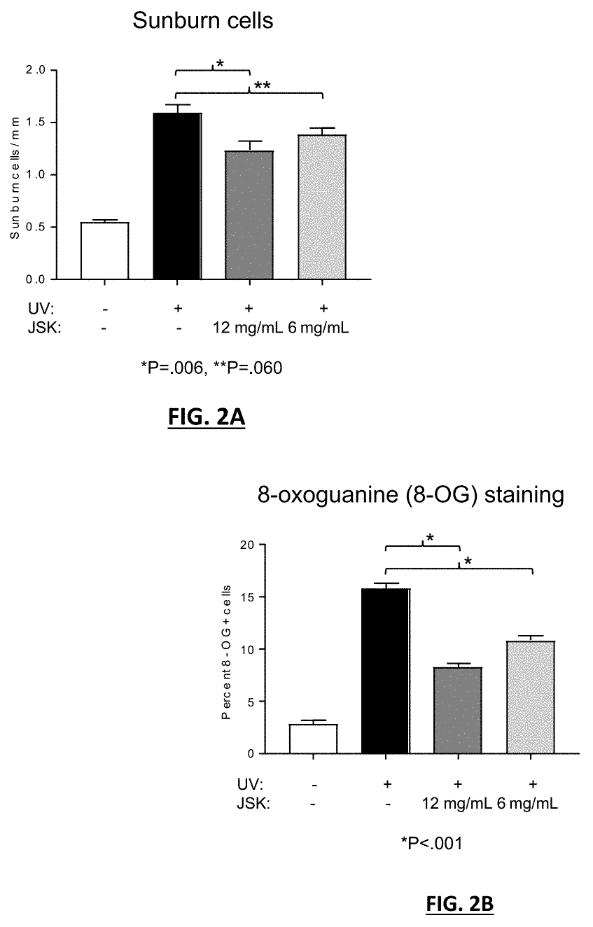

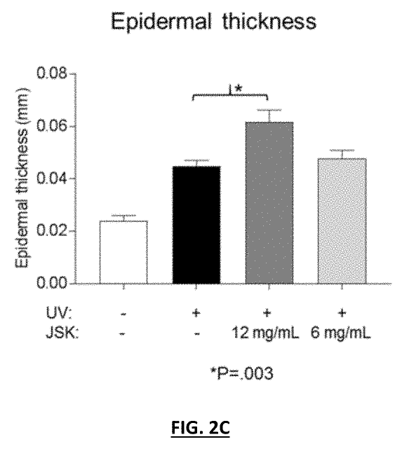

H&E staining was used (FIG. 2A) to evaluate the count of Sunburn Cells, which are keratinocytes undergoing UV-induced apoptosis. Cells from JS-K treated mice exhibited a moderate decrease in the number of Sunburn Cells with a dose effect. The differences with controls were statistically significant for the higher dose of JS-K (12 mg/mL).

FIG. 2B shows the percent of cells positive for 8-oxoguanine (8-OG) as evaluated by immunohistochemistry. 8-OG is a marker of oxydative damage. Compared to the controls fewer cells from the mice treated with JS-K were positive for 8-OG, with a dose effect. Differences from the controls were statistically significant for both doses of JS-K. There was no evidence of toxicity, skin irritation, skin ulceration, or infection with the treatments. The results indicate that JS-K may be protective against UV-induced cellular changes.

FIG. 2C shows a dose dependent JS-K effect on epidermal thickness for cells exposed to UV light, with the 12 mg/mL JS-K having a markedly greater effect than the 6 mg/mL JS-K.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A composition for the treatment of a skin condition including actinic keratosis and cancer, the composition comprising O2-(2,4-dinitrophenyl)1-[(4-ethoxycarbonyl)piperazin-1-yl]diazen-1-ium-1,2-diolate, (JS-K).

2. The composition of claim 1, further comprising a lipophilic carrier.

3. The composition of claim 2, wherein the concentration of JS-K is in the range from about 0.5 mg/mL to about 25 mg/mL.

4. The composition of claim 3, wherein the concentration of JS-K is from 1 to 2 mg/mL, from 2 to 3 mg/mL, from 3 to 4 mg/mL, from 4 to 5 mg/mL, from 5 to 6 mg/mL, from 6 to 7 mg/mL, from 7 to 8 mg/mL, from 8 to 9 mg/mL, from 9 to 10 mg/mL, from 10 to 11 mg/mL, from 11 to 12 mg/mL, from 12 to 13 mg/mL, from 13 to 14 mg/mL, from 14 to 15 mg/mL, from 15 to 16 mg/mL, from 16 to 17 mg/mL, from 17 to 18 mg/mL, from 18 to 19 mg/mL, from 19 to 20 mg/mL, from 20 to 21 mg/mL, from 21 to 22 mg/mL, from 22 to 23 mg/mL, from 23 to 24 mg/mL, or from 24 to 25 mg/mL.

5. The composition of claim 4, wherein the concentration of JS-K is 12 mg/mL.

6. The composition of claim 2, wherein the lipophilic carrier comprises at least one of a hydrocarbon, a vegetable oil, an animal fat, a silicone, an alcohol, an acid, a combination of lipophilic excipients, a self-emulsifying formula, a polyglycolyzed glyceride, a polymeric excipient, a surfactant, a pluronic micellar formulation, a cyclodextrin, a polyoxyether, and other carriers.

7. The composition of claim 6, formulated as at least one of a lotion, a gel, a powder, a paste, an ointment, a wax, an oil, a lipid, a lipid containing vesicle, an anhydrous absorption paste, an oil-in-water or water-in-oil emulsion, a carbowax, a polyethelene glycol, a lipophilic skin emollient, a penetration enhancer and a semisolid gel and a semi-solid mixture.

8. The composition of claim 2, wherein the composition comprises a sustained-release formula.

9. The sustained-release formula of claim 8, further comprising semipermeable matrices of solid hydrophobic polymers and wherein the matrices comprise at least one of a shaped article, a film, a microcapsule, a polyester, a hydrogel, a copolymer of L-glutamic acid and gamma ethyl-L-glutamate, a non-degradable ethylene-vinyl acetate, and a degradable lactic acid-glycolic acid copolymer.

10. A method for the treatment of a skin condition, the method comprising applying to a patient in need of treatment a composition comprising O2-(2,4-dinitrophenyl) 1-[(4-ethoxycarbonyl)piperazin-1-yI] diazen-1-ium-1,2-diolate.

11. The method of claim 10, wherein a daily dose is in the range of about 0.5 mg to about 200 mg.

12. The method of claim 11, wherein the daily dose is from 1 to 5 mg, from 5 to 10 mg, from 10 to 20 mg, from 20 to 30 mg, from 30 to 40 mg, from 40 to 50 mg, from 50 to 60 mg, from 60 to 70 mg, from 70 to 80 mg, from 80 mg to 90 mg, from 90 to 100 mg, from 100 to 125 mg, from 125 to 150 mg, from 150 to 1785 mg, or from 175 to 200 mg.

13. The method of claim 10, wherein a route of administration is at least one of topical, transdermal, cutaneous, subcutaneous, mucosal, and transmucosal.

14. The method of claim 11, wherein the daily dose is administered once daily.

15. The method of claim 11, wherein the daily dose is administered two or more times daily.

16. The method of claim 10, wherein a daily dose is administered as a sustained release formula.

17. The method of claim 10, wherein the skin condition comprises actinic keratosis or cancer.

18. The method of claim 17, wherein the cancer is at least one of squamous cell skin cancer, basal cell carcinoma, Merkel cell carcinoma, lymphoma of the skin, and melanoma skin cancer.

19. The method of claim 10, wherein the patient in need of treatment is at least one of a mammal, a bird, a reptile, an amphibian, and a fish.

20. The method of claim 19, wherein the mammal is a human.
